(19) United States
(12) Patent Application Publication
(10) Pub. No.: US 2026/0216163 A1
(43) Pub. Date: Jul. 30, 2026

(54) CANCER COMBINATION THERAPY INCLUDING A FLT3-INHIBITOR

(71) Applicants: RYVU THERAPEUTICS S.A., Kraków (PL); BERLIN-CHEMIE AG, Berlin (DE)

(72) Inventors: Monica BINASCHI, Pomezia (IT); Daniela BELLAROSA, Pomezia (IT); Giuseppe MERLINO, Pomezia (IT); Krzysztof BRZOZKA, Kraków (PL)

(21) Appl. No.: 18/998,838

(22) PCT Filed: Jul. 28, 2024
(86) PCT No.: PCT/EP2023/070945

§ 371 Date: Jan. 27, 2025

(30) Foreign Application Priority Data
Jul. 29, 2022 (EP) 22460035.3

(51) Int. Cl. A61K 31/454; A61K 31/497; A61K 31/5377; A61K 31/553; A61P 35/02

(52) U.S. Cl. CPC A61K 31/454; A61K 31/497; A61K 31/5377; A61K 31/553; A61P 35/02

(57) ABSTRACT

The present invention is inter alia concerned with a combination of (i) a FLT3-inhibitor and (ii) SEL24/MEN1703 for use as medicament, preferably for use in the treatment of a patient suffering from cancer. The present invention is also concerned with a kit of dosage forms comprising (i) a dosage form comprising a FLT3-inhibitor and (ii) a dosage form comprising SEL24/MEN1703; as well as dosage form comprising (i) a FLT3-inhibitor and (ii) SEL24/MEN1703.

### FIELD OF THE INVENTION

The present invention is in the field of cancer therapy. More specifically, the present invention is directed in one aspect to a combination of (i) a FLT3-inhibitor and (ii) SEL24/MEN1703 for use as medicament. In another aspect, the present invention is concerned with a combination of (i) a FLT3-inhibitor and (ii) SEL24/MEN1703 for use in the treatment of a patient suffering from cancer. In yet another aspect, the present invention is directed to a kit of dosage forms comprising (i) a dosage form comprising a FLT3-inhibitor and (ii) a dosage form comprising SEL24/MEN1703. In yet another aspect, the present invention is concerned with a dosage form comprising (i) a FLT3-inhibitor and (ii) SEL24/MEN1703.

### BACKGROUND OF THE INVENTION

FLT3-mutations in AML patients are known for more than 25 years now, with the most common FLT3-ITD (“internal tandem duplication”) mutation reported first in 1996 (see Nakao M et al., 1996). Such mutations result in a constitutively active FLT3, which has kinase activity. Accordingly, FLT3-inhibitors were subsequently developed aiming at treating the disease, with clinical trials of FTL3-inhibitors in AML enrolling their first patients in 2002, as outlined in Levis and Perl, 2020. The review by Kennedy and Smith, 2020, summarizes the understanding of FLT3 mutations in AML and inter alia provides an overview of established FTL3-inhibitors (see Table 1 therein, where first and second generation inhibitors are given and wherein the type of inhibition is given) as well as clinical trials of established FLT3-inhibitors in newly diagnosed AML (see Table 2 therein). Kennedy and Smith, 2020, further summarize clinical trials of established FLT3-inhibitors in combination therapy (see Table 4 therein) or of novel FLT3-inhibitors including inhibitors with dual inhibitory activity (“dual agents”, see Table 5 therein). The review by Yuan et al., 2019, focuses on such dual FLT3-inhibitors.

The first approved FLT3-inhibitor was Midostaurin, which was approved in patients with FLT3-mutated AML (Rydapt®, approved in the US and Europe in 2017). Further FLT3-inhibitors were tested, including Quizartinib. The use of Quizartinib showed a limitation that can occur during the treatment, namely resistance due to on-target kinase-activating FLT3-mutations, which often occurred within two to four months of initiating Quizartinib.

Accordingly, there is still the need to improve the initial or “first generation” FLT3-inhibitors.

Taking into account the results of the initial FLT 3-inhibitors, further inhibitors were and are being developed, including Gilteritinib. Clinical studies with Gilteritinib showed that a therapy with Gilteritinib was more effective and less toxic than standard chemotherapy. Subsequently, a Gilteritinib-containing product received marketing authorizations for AML-patients with a FLT3-mutation inter alia in the US and Europe (Xospata®). It is noteworthy that Xospata© was approved by the FDA for the respective patient population upon proof of superiority to existing therapies. Gilteritinib is administered in Xospata® as monotherapy.

As there is, however, still resistance to Gilteritinib and as the responses to Gilteritinib monotherapy may thus be short-lived, there is still a need for improving the therapy, and first clinical trials investigating combinations of Gilteritinib e.g. with intensive induction chemotherapy or lower-toxicity agents have been initiated and show promising results (see Levis and Perl, 2020). A further example for such a combination is the combination of CUDC-907, a dual inhibitor of PI3K and histone deacetylases, and Gilteritinib, which shows promising in vitro and in vivo antileukemic activity against FLT3-ITD AML (see Qiao et al. 2021). Also, Gilteritinib is studied as additional active to be added to a different backbone, e.g. in patients unresponsive to venetoclax plus azacitidine (see Zhang et al., 2022), where gilteritinib was administered to such an unresponsive patient together with venetoclax and azacitidine (induction therapy, wherein the maintenance therapy was venetoclax and Gilteritinib) with a promising outcome.

Accordingly, there is still the need to improve the more recent or “second generation” FLT3-inhibitors, in particular Gilteritinib.

### OBJECTS AND SUMMARY OF THE INVENTION

The inventors of the present invention have surprisingly found that the combination of a FLT3-inhibitor and SEL24/MEN1703 acts synergistically and therefore corresponds to a very promising new combination therapy in particular in patients suffering from acute myeloid leukemia (AML).

In a first aspect, the present invention is directed to a combination of (i) a FLT3-inhibitor and (ii) SEL24/MEN1703 for use as medicament.

In a preferred embodiment, the FLT3-inhibitor is a Type I FLT3-inhibitor. The Type I FLT3-inhibitor may be selected from the group consisting of Midostaurin, Gilteritinib, Lestaurtinib and Crenolanib.

In another preferred embodiment, the FLT3-inhibitor is a Type II FLT3-inhibitor. The Type II FLT3-inhibitor may be selected from the group consisting of Quizartinib and Sorafenib.

In a preferred embodiment, the FLT3-inhibitor is selected from the group consisting of Gilteritinib, Quizartinib, Midostaurin, and combinations thereof. It can be preferred that the FTL3-inhibitor is Gilteritinib.

In an embodiment, SEL24/MEN1703 is administered at a daily dose of about 50 mg to about 150 mg. It is preferred that SEL24/MEN1703 is administered at a daily dose of about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg. It is most preferred that SEL24/MEN1703 is administered at a daily dose of about 100 mg, about 105 mg, about 110 mg, about 115 mg, about 120 mg or about 125 mg. It is preferred that SEL24/MEN1703 is administered once a day. It is further preferred that SEL24/MEN1703 is administered orally.

In an embodiment, Gilteritinib is administered at a daily dose of about 40 mg to about 400 mg. It is preferred that Gilteritinib is administered at a daily dose of about 40 mg, about 80 mg, about 120 mg, or about 240 mg. It is most preferred that Gilteritinib is administered at a daily dose of about 100 mg, about 105 mg, about 110 mg, about 115 mg or about 120 mg. It is preferred that Gilteritinib is administered once a day. It is further preferred that Gilteritinib is administered orally.

In an embodiment, Quizartinib is administered at a daily dose of about 10 mg to about 50 mg. It is preferred that Quizartinib is administered at a daily dose of about 10 mg, about 20 mg, about 30 mg, about 40 mg, or about 50 mg. It is most preferred that Quizartinib is administered at a daily dose of about 15 mg, about 17 mg, about 20 mg, or about 25 mg. It is preferred that Quizartinib is administered once a day. It is further preferred that Quizartinib is administered orally.

In an embodiment, Midostaurin is administered at a daily dose of about 20 mg to about 80 mg. It is preferred that Midostaurin is administered at a daily dose of about 20 mg, about 30 mg, about 40 mg, about 50 mg, or about 60 mg. It is most preferred that Midostaurin is administered at a daily dose of about 35 mg, about 40 mg, about 45 mg, or about 50 mg. It is preferred that Midostaurin is administered twice a day. It is further preferred that Midostaurin is administered orally.

In an embodiment, the combination for use of the first aspect does not comprise the additional administration of chemotherapy.

In an embodiment relating to the combination for use as medicament, (i) and (ii) are administered as separate dosage forms. In this embodiment, the administration may be concomitantly or sequentially. In yet an alternative embodiment relating to the combination for use as medicament, (i) and (ii) are administered together in a dosage form.

In a second aspect, the present invention is directed to a combination of (i) a FLT3-inhibitor and (ii) SEL24/MEN1703 for use in the treatment of a patient suffering from cancer.

In a preferred embodiment, the (i) FLT3-inhibitor is a Type I FLT3-inhibitor. The Type I FLT3-inhibitor may be selected from the group consisting of Midostaurin, Gilteritinib, Lestaurtinib and Crenolanib.

In another preferred embodiment, the (i) FLT3-inhibitor is a Type II FLT3-inhibitor. The Type II FLT3-inhibitor may be selected from the group consisting of Quizartinib and Sorafenib.

In a preferred embodiment, the FLT3-inhibitor is selected from the group consisting of Gilteritinib, Quizartinib, Midostaurin, and combinations thereof. It can be preferred that the FTL3-inhibitor is Gilteritinib.

In a preferred embodiment, the cancer is a hematological cancer. In an even more preferred embodiment, the cancer is a leukemia. In a most preferred embodiment, the cancer is AML. The combination for use of the second aspect can be a first-line treatment of AML. However, the combination for use of the second aspect may alternatively be used to treat patients that progress over a prior line of treatment(s). In one embodiment, the combination of the invention may be used to treat relapsed or recurrent AML, or refractory AML.

When the patient is suffering from AML, it is preferred that the patient suffering from AML exhibits a FLT3 mutation that results in overactivation of FLT3 signalling. The mutation in the FLT3 may even result in constitutively active FLT3 signalling (in the meaning that the signaling activity of FLT3 is constitutively active). The FLT3 mutation is caused by at least one base mutation in the FLT3 gene, resulting in the afore-mentioned FLT3 mutation on a protein level that results in overactivation of FLT3 signaling. Such mutations are known in the field, as mentioned in the background section above. Preferably, the FLT3 mutation is a FLT3-ITD mutation, a FLT3-TKD mutation, or a combination of a FLT3-ITD mutation and FLT3-TKD mutation. Additionally or alternatively, the patient suffering from cancer, including a patient suffering from AML, may exhibit at least one IDH1 and/or IDH2 mutation, preferably at least two IDH1 and/or IDH2 mutations. Additionally or alternatively, the patient suffering from cancer may be unfit for a chemotherapy.

When the patient is suffering from AML, the patient may, however, also exhibit a FLT3 wild-type sequence. Additionally or alternatively, the patient suffering from cancer may be unfit for a chemotherapy.

In an embodiment, SEL24/MEN1703 is administered at a daily dose of about 50 mg to about 150 mg. It is preferred that SEL24/MEN1703 is administered at a daily dose of about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg. It is most preferred that SEL24/MEN1703 is administered at a daily dose of about 100 mg, about 105 mg, about 110 mg, about 115 mg, about 120 mg or about 125 mg. It is preferred that SEL24/MEN1703 is administered once a day. It is further preferred that SEL24/MEN1703 is administered orally.

In an embodiment, Gilteritinib is administered at a daily dose of about 40 mg to about 400 mg. It is preferred that Gilteritinib is administered at a daily dose of about 40 mg, about 80 mg, about 120 mg, or about 240 mg. It is most preferred that Gilteritinib is administered at a daily dose of about 100 mg, about 105 mg, about 110 mg, about 115 mg or about 120 mg. It is preferred that Gilteritinib is administered once a day. It is further preferred that Gilteritinib is administered orally.

In an embodiment, Quizartinib is administered at a daily dose of about 10 mg to about 50 mg. It is preferred that Quizartinib is administered at a daily dose of about 10 mg, about 20 mg, about 30 mg, about 40 mg, or about 50 mg. It is most preferred that Quizartinib is administered at a daily dose of about 15 mg, about 17 mg, about 20 mg, or about 25 mg. It is preferred that Quizartinib is administered once a day. It is further preferred that Quizartinib is administered orally.

In an embodiment, Midostaurin is administered at a daily dose of about 20 mg to about 80 mg. It is preferred that Midostaurin is administered at a daily dose of about 20 mg, about 30 mg, about 40 mg, about 50 mg, or about 60 mg. It is most preferred that Midostaurin is administered at a daily dose of about 35 mg, about 40 mg, about 45 mg, or about 50 mg. It is preferred that Midostaurin is administered twice a day. It is further preferred that Midostaurin is administered orally.

In an embodiment, the combination for use in the second aspect, in particular the combination for use in the treatment of AML, does not comprise the additional administration of chemotherapy.

In an embodiment relating to the combination for use in the treatment of a patient suffering from cancer, (i) and (ii) are administered as separate dosage forms. In this embodiment, the administration may be concomitantly or sequentially. In yet an alternative embodiment relating to the combination for use in the treatment of a patient suffering from cancer, (i) and (ii) are administered together in a dosage form.

In a preferred embodiment, the FLT3-inhibitor is Gilteritinib, the cancer is AML, SEL24/MEN1703 is administered at a daily dose of about 80 mg to about 120 mg, preferably orally, and Gilteritinib is administered at a daily dose of about 100 mg to about 115 mg, preferably orally. It can be preferred in this embodiment that the patient suffering from AML exhibits a FLT3 mutation and, optionally, exhibits at least one IDH1 and/or IDH2 mutation.

In a third aspect, the present invention is directed to a kit of dosage forms comprising (i) a dosage form comprising a FLT3-inhibitor and (ii) a dosage form comprising SEL24/MEN1703.

In a preferred embodiment, the FLT3-inhibitor comprised in dosage form (i) is a Type I FLT3-inhibitor. The Type I FLT3-inhibitor may be selected from the group consisting of Midostaurin, Gilteritinib, Lestaurtinib and Crenolanib.

In another preferred embodiment, the FLT3-inhibitor comprised in dosage form (i) is a Type II FLT3-inhibitor. The Type II FLT3-inhibitor may be selected from the group consisting of Quizartinib and Sorafenib.

In a preferred embodiment, the FLT3-inhibitor is selected from the group consisting of Gilteritinib, Quizartinib, Midostaurin, and combinations thereof. It can be preferred that the FTL3-inhibitor is Gilteritinib.

In an embodiment, the dosage form comprising SEL24/MEN1703 comprises SEL24/MEN1703 in an amount of about 50 mg to about 150 mg, preferably in an amount of about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg, most preferably in an amount of about 100 mg, about 105 mg, about 110 mg, about 115 mg, about 120 mg or about 125 mg. It is preferred that the dosage form comprising SEL24/MEN1703 is a once-a-day dosage form. It is further preferred that the dosage form comprising SEL24/MEN1703 is an oral dosage form.

In an embodiment, the dosage form comprising Gilteritinib as FLT3-inhibitor comprises Gilteritinib in an amount of about 40 mg to about 400 mg, preferably about 40 mg, about 80 mg, about 120 mg, or about 240 mg, most preferably about 100 mg, about 105 mg, about 110 mg, about 115 mg or about 120 mg. It is preferred that the dosage form comprising Gilteritinib as FLT3-inhibitor is a once-a-day dosage from. It is further preferred that the dosage form comprising Gilteritinib as FLT3-inhibitor is an oral dosage form.

In an embodiment, the dosage form comprising Quizartinib as FLT3-inhibitor Quizartinib comprises Quizartinib in an amount of about 10 mg to about 50 mg, preferably about 10 mg, about 20 mg, about 30 mg, about 40 mg, or about 50 mg, most preferably about 15 mg, about 17 mg, about 20 mg, or about 25 mg. It is preferred that the dosage form comprising Quizartinib as FLT3-inhibitor is a once-a-day dosage form. It is further preferred that the dosage form comprising Quizartinib as FLT3-inhibitor is an oral dosage form.

In an embodiment, the dosage form comprising Midostaurin as FLT3-inhibitor comprises Midostaurin in an amount of about 20 mg to about 80 mg, preferably about 20 mg, about 30 mg, about 40 mg, about 50 mg, or about 60 mg, most preferably about 35 mg, about 40 mg, about 45 mg, or about 50 mg. It is preferred that the dosage form comprising Midostaurin is a twice-a-day dosage form. It is further preferred that the dosage form comprising Midostaurin is an oral dosage form.

Each dosage form typically contains at least one pharmaceutically acceptable excipient as defined in section 2 of the detailed description below.

In an embodiment, the kit of the third aspect further comprises a leaflet setting out the instructions how to use and administer the dosage forms.

In a fourth aspect, the present invention is directed to a dosage form comprising (i) a FLT3-inhibitor and (ii) SEL24/MEN1703.

In a preferred embodiment, the FLT3-inhibitor comprised in the dosage form is a Type I FLT3-inhibitor. The Type I FLT3-inhibitor may be selected from the group consisting of Midostaurin, Gilteritinib, Lestaurtinib and Crenolanib.

In another preferred embodiment, the FLT3-inhibitor comprised in the dosage form is a Type II FLT3-inhibitor. The Type II FLT3-inhibitor may be selected from the group consisting of Quizartinib and Sorafenib.

In a preferred embodiment, the FLT3-inhibitor is selected from the group consisting of Gilteritinib, Quizartinib, Midostaurin, and combinations thereof. It can be preferred that the FTL3-inhibitor is Gilteritinib.

In an embodiment, the dosage form comprising SEL24/MEN1703 comprises SEL24/MEN1703 in an amount of about 50 mg to about 150 mg, preferably in an amount of about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg, most preferably in an amount of about 100 mg, about 105 mg, about 110 mg, about 115 mg, about 120 mg or about 125 mg.

If the dosage form comprises Gilteritinib as FLT3-inhibitor, the dosage form comprises in an embodiment Gilteritinib in an amount of about 40 mg to about 400 mg, preferably about 40 mg, about 80 mg, about 120 mg, or about 240 mg, most preferably about 100 mg, about 105 mg, about 110 mg, about 115 mg or about 120 mg.

If the dosage form comprises Quizartinib as FLT3-inhibitor, the dosage form comprises Quizartinib in an amount of about 10 mg to about 50 mg, preferably about 10 mg, about 20 mg, about 30 mg, about 40 mg, or about 50 mg, most preferably about 15 mg, about 17 mg, about 20 mg, or about 25 mg.

If the dosage form comprises Midostaurin as FLT3-inhibitor, the dosage form comprises Midostaurin in an amount of about 20 mg to about 80 mg, preferably about 20 mg, about 30 mg, about 40 mg, about 50 mg, or about 60 mg, most preferably about 35 mg, about 40 mg, about 45 mg, or about 50 mg.

In an embodiment, the dosage form is a once-a-day or a twice-a-day dosage form. It is further preferred that the dosage form is an oral dosage form.

The dosage form typically contains at least one pharmaceutically acceptable excipient as defined in section 2 of the detailed description below.

In an embodiment, the dosage form of the fourth aspect comes with a leaflet setting out the instructions how to use and administer the dosage form.

In a fifth aspect, the present invention is directed to a method of treating cancer in a patient in need thereof, said method comprising administering to the patient an effective amount of (i) a FLT3-inhibitor and an effective amount of (ii) SEL24/MEN1703.

All embodiments outlined above for the second aspect equally apply for the fifth aspect.

### DESCRIPTION OF THE FIGURES

FIG. 1: Statistical analysis of cytotoxicity data at IC75 concentrations in MV4-11 cells (“men”=MEN, “gilt”=Gilt, “men+gilt”=“MEN+Gilt”). The MEN+Gilt combination induced a cytotoxicity significantly different from the Gilteritinib-induced cytotoxicity (Student's t test, p=0.0331).

FIG. 2: In vivo studies in an AML MV4-11 cell line xenograft. Tumor cells were injected s.c. into SCID mice at day 0 and the treatments using either MEN (“MEN1703”), Gilteritinib, the MEN+Gilt combination or a control (vehicle only) started on day 19. The first arrows underneath the x-axis show the MEN-dosing (from day 19 to day 32), whereas the second arrows underneath the x-axis show the Gilt-dosing (from day 19 to day 40). Statistical analysis was performed according to the Mann-Whitney rank test, evaluated at day 33 and day 40. Tumor growth inhibition with the combination of MEN+Gilt is significantly different from the single treatments and the control groups. (A) shows the tumor volume for the single as well as the combination treatments over time, whereas (B) shows the tumor volume at day 33.

FIG. 3: In vivo studies in an AML MOLM-13 cell line xenograft. Tumor cells were injected s.c. into SCID mice at day 0 and the drug dosing using either MEN (“MEN1703”), Gilteritinib, the MEN+Gilt combination or a control (vehicle only) started on day 15. The first arrows underneath the x-axis show the MEN-dosing (from day 15 to day 28), whereas the second arrows underneath the x-axis show the Gilt-dosing (from day 15 to day 28). Statistical analysis was performed according to the Mann-Whitney rank test, evaluated at day 29. Tumor growth inhibition with the combination of MEN+Gilt is significantly different from the control group. (A) shows the tumor volume for single as well as the combination treatments over time, whereas (B) shows the tumor volume at day 29.

FIG. 4: Statistical analysis of cytotoxicity data in MOLM13 cell line (A) at IC50 concentrations and (B) at IC75 concentrations (“Quiz”=quizartinib, “MEN1703+Quiz”=“MEN1703+Quizartinib”). The MEN1703+Quiz combination induced a cytotoxicity significantly different from the Quizartinib-induced cytotoxicity (Tukey's multiple comparison one-way ANOVA test; p<0.05*).

FIG. 5: Statistical analysis of cytotoxicity data in FLT3 wt KG1 cell line (A) at MEN1703 IC50 and Gilteritinib IC75 concentrations and (B) at MEN1703 IC25 and Gilteritinib IC75 concentrations. The MEN1703 IC50+Gilteritinib IC75 and MEN1703 IC25+Gilteritinib IC75 combinations induced a cytotoxicity significantly different from MEN1703 as single agent (Tukey's multiple comparison one-way ANOVA test; p=0.0158*, p=0.0005*** respectively).

### DETAILED DESCRIPTION OF THE INVENTION

Before the present invention is described in more detail, the following definitions are introduced.

### 1. Definitions

As used in the specification and the claims, the singular forms of “a” and “an” also include the corresponding plurals unless the context clearly dictates otherwise.

The term “about” in the context of the present invention denotes an interval of accuracy that a person skilled in the art will understand to still ensure the technical effect of the feature in question. The term typically indicates a deviation from the indicated numerical value of ±10% and preferably ±5%.

It needs to be understood that the term “comprising” is not limiting. For the purposes of the present invention, the term “consisting of” is considered to be a preferred embodiment of the term “comprising”. If hereinafter a group is defined to comprise at least a certain number of embodiments, this is also meant to encompass a group which preferably consists of these embodiments only.

The term “a combination” or “in combination with” as used herein is not intended to imply that the therapy or the active agents (i) and (ii) must be administered at the same time and/or formulated for delivery together, although such therapy and formulations are within the scope of the present invention. The active agents in the combination can be administered concurrently with, prior to, or subsequent to, each other, and even one or more other additional therapies or active agents. The active agents or therapeutic protocol can be administered in any order. In general, each active agent will be administered at a dose and/or on a time schedule determined for that active agent. Further, in general, it is expected that active agents used in combination are used at doses that do not exceed the doses at which they are used individually. In some embodiments, the doses used in the combination will be lower than the doses used individually. In some embodiments, one of the two active agents is administered at a therapeutic or lower-than therapeutic dose, e.g. the FLT3-inhibitor is administered at a lower-than therapeutic dose or SEL24/MEN1703 is administered at a lower-than therapeutic dose (wherein the “lower-than therapeutic dose” is derived from a comparison to the therapeutic dose as single active agent in a monotherapy). A lower-than therapeutic dose can e.g. be 10-20%, 20-30%, 30-40%, 40-50%, 50-60%, 60-70%, 70-80%, or 80-90% lower than the corresponding monotherapy.

The term “FLT3” as used herein refers to the receptor-type tyrosine-protein kinase FLT3 (also known as “Cluster of differentiation antigen 135”, “CD135”, “fms like tyrosine kinase 3”, “fetal liver kinase-2” or “Flk2”), a protein encoded in humans by the FLT3 gene. FLT3 is a cytokine receptor which belongs to the receptor tyrosine kinase class Ill.

The term “FLT3-inhibitor” as used herein means a compound capable of inhibiting the kinase FLT3, in particular inhibiting a mutated version of FLT3, which results in an overactivation of FLT3 signaling, e.g. constitutively active FLT3. Such compounds are known in the field, in particular when it comes to AML treatment (see Levis and Perl, 2020). Typically, such compounds are small molecules that strongly (and, as the case may be [i.e. not necessarily] selectively) bind to FLT3 such that FLT3 is inhibited. FLT3-inhibitors can be subdivided based upon how they interact with the intracellular kinase domain of the FLT3 receptor. In normal physiology, FLT3 ligand binds to the extracellular domain, causing the FLT3 receptor to dimerize, assume an enzymatically active conformation and subsequently activate downstream signaling. Type I FLT3-inhibitors bind the receptor in the active conformation, thus inhibiting both FLT3-TKD and FLT3-ITD mutated receptors. Type II FLT3-inhibitors bind to a region adjacent to the ATP-binding pocket and only inhibit the receptor in the inactive conformation. Type II inhibitors are inactive against most FLT3-TKD mutations as these mutations bias the active kinase conformation of FLT3 (see also Kennedy and Smith, 2020, passage bridging pages 3 and 4).

The term “FLT3-ITD mutation” as used herein means a FLT3 “internal tandem duplication, ITD” mutation. The term “FLT3-TKD mutation” as used herein means a FLT3 “tyrosine kinase domain” mutation.

The term “SEL24/MEN1703” (alternatively referred to herein as “MEN”, “Men”, “men” or “Men1703”) as used herein means the compound 5,6-dibromo-4-nitro-2-(piperidin-4-yl)-1-(propan-2-yl)-1H-1,3-benzodiazole, in the form of the free base or a pharmaceutically acceptable salt thereof (such as the HCl-salt). The free base form has the CAS-number 1616359-00-2, whereas the HCl-salt form has the CAS-number 2769008-22-0. The compound is a dual pan-PIM/FLT3 inhibitor, which has inter alia been shown to inhibit the growth of a broad panel of AML cell lines in xenograft models. The rationale for the development of this dual inhibitor was that PIM kinases are deemed to be major drivers of the resistance to FLT3-inhibitors. SEL24/MEN1703 is characterized in more detail e.g. in Czardybon et al., 2018. WO 2014/096388 discloses SEL24/MEN1703 as Compound 26A therein and characterizes SEL24/MEN1703 as dual pan-PIM/FLT3 inhibitor, see Table 1A of WO 2014/096388. WO 2014/096388 fails to disclose a combination of SEL24/MEN1703 with a FLT3-inhibitor, let alone a combination of any of the compound within the scope of WO 2014/096388 with a FLT3-inhibitor.

The term “Gilteritinib” as used herein mean the FLT3-inhibitor Gilteritinib, marketed under the tradename Xospata©, which is authorized for AML-treatment. Further details about Gilteritinib can inter alia be found in the product leaflet or the regulatory dossiers.

The term “Quizartinib” as used herein means the FLT3-inhibitor Quizartinib, for which a marketing authorization was applied for in Europe by Daiichi Sankyo Europe GmbH under the name “Vanflyta®”. The EMA issued an opinion in 2019 recommending the refusal of the marketing authorization because the benefits of Quizartinib treatment did in the EMA's opinion not outweigh its risks.

The term “Midostaurin” as used herein means the FLT3-inhibitor Midostaurin, marketed under the tradename Rydapt®, which is authorized for AML-treatment, wherein at the start of treatment, Rydapt® must always be used together with chemotherapy. Further details about Midostaurin can inter alia be found in the product leaflet or the regulatory dossiers.

The term “overactivation” of the FLT3 as used herein means that the FLT3 is more active compared to the wild-type situation, in particular more active with respect to downstream activation and signaling, thus resulting in cancerous cell growth.

The term “small molecule” as used herein refers to a small organic compound having a low molecular weight. A small molecule in the context of the present invention preferably has a molecular weight of less than 5000 Dalton, more preferably of less than 4000 Dalton, more preferably less than 3000 Dalton, more preferably less than 2000 Dalton or even more preferably less than 1000 Dalton. In a particularly preferred embodiment a small molecule in the context of the present invention has a molecular weight of less than 800 Dalton. In another preferred embodiment, a small molecule in the context of the present invention has a molecular weight of 50 to 3000 Dalton, preferably of 100 to 2000 Dalton, more preferably of 100 to 1500 Dalton and even more preferably of 100 to 1000 Dalton.

The term “treatment” as used herein refers to clinical intervention in order to cure or ameliorate a disease, prevent recurrence of a disease, alleviate symptoms of a disease, diminish any direct or indirect pathological consequences of a disease, achieve a stabilized (i.e., not worsening) state of disease, prevent metastasis, decrease the rate of disease progression, and/or prolong survival as compared to expected survival if not receiving treatment.

The term “relapsed or recurrent AML” as used herein means that the AML has come back after treatment (using a different drug, optionally a drug combination, as the combination of the present invention) and remission.

The term “refractory AML” as used herein means that the leukemia did not respond to previous treatment (using a different drug, optionally a drug combination, as the combination of the present invention).

The terms “IDH1 and/or IDH2 mutation” as used herein means a mutation in the “isocitrate dehydrogenase 1” gene or the “isocitrate dehydrogenase 2” gene, which encode the corresponding isocitrate dehydrogenases.

### 2. Pharmaceutical Compositions

The “FLT3-inhibitor” and “SEL24/MEN1703” are “pharmaceutically active agents” or “active agents” for the purposes of the present invention. As noted above, they may either be present in separate dosage forms or comprised in a single dosage form.

“Pharmaceutically active agents” as used herein means that the compounds are potent of modulating a response in a patient, i.e. a human or animal being in vivo. The term “pharmaceutically acceptable excipient” as used herein refers to excipients commonly comprised in pharmaceutical dosage forms or compositions, which are known to the skilled person. Such excipients are exemplary listed below. In view of the definition “pharmaceutically active agents” as given above, a pharmaceutically acceptable excipient can be defined as being pharmaceutically inactive.

If a marketed FLT3-inhibitor is used in combination with SEL24/MEN1703, it is typically preferred that the administration occurs via separate dosage forms and that the FLT3-inhibitor is administered in the dosage form and via the administration route that is authorized. SEL24/MEN1703 may be administered in a dosage form as set out in the following or in a dosage form in which it is currently undergoing clinical testing.

A dosage form for use according to the present invention may be formulated for oral, buccal, nasal, rectal, topical, transdermal or parenteral application. Oral application is particularly preferred. Parenteral application includes intravenous, intramuscular or subcutaneous administration. A dosage form of the present invention may also be designated as formulation or pharmaceutical composition.

In general, a pharmaceutical composition according to the present invention can comprise various pharmaceutically acceptable excipients which will be selected depending on which functionality is to be achieved for the composition. A “pharmaceutically acceptable excipient” in the meaning of the present invention can be any substance used for the preparation of pharmaceutical dosage forms, including coating materials, film-forming materials, fillers, disintegrating agents, release-modifying materials, carrier materials, diluents, binding agents and other adjuvants. Typical pharmaceutically acceptable excipients include substances like sucrose, mannitol, sorbitol, starch and starch derivatives, lactose, and lubricating agents such as magnesium stearate, disintegrants and buffering agents.

The term “carrier” denotes pharmaceutically acceptable organic or inorganic carrier substances with which the active ingredient is combined to facilitate the application. Suitable pharmaceutically acceptable carriers include, for instance, water, salt solutions, alcohols, oils, preferably vegetable oils, polyethylene glycols, gelatin, lactose, amylose, magnesium stearate, surfactants, perfume oil, fatty acid monoglycerides and diglycerides, petroethral fatty acid esters, hydroxymethyl-cellulose, polyvinylpyrrolidone and the like. The pharmaceutical compositions can be sterilized and if desired, mixed with auxiliary agents, like lubricants, preservatives, stabilizers, wetting agents, emulsifiers, salts for influencing osmotic pressure, buffers, colorings, flavoring and/or aromatic substances and the like which do not deleteriously react with the active compound.

If liquid dosage forms are considered for the present invention, these can include pharmaceutically acceptable emulsions, solutions, suspensions and syrups containing inert diluents commonly used in the art such as water. These dosage forms may contain e.g. microcrystalline cellulose for imparting bulk, alginic acid or sodium alginate as a suspending agent, methylcellulose as a viscosity enhancer and sweeteners/flavouring agents.

For parenteral application, particularly suitable vehicles consist of solutions, preferably oily or aqueous solutions, as well as suspensions, emulsions, or implants. Pharmaceutical formulations for parenteral administration are particularly preferred and include aqueous solutions in water-soluble form. Additionally, suspensions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran.

For injectable preparations, sterile injectable aqueous or oleaginous suspensions can for example be formulated according to the known art using suitable dispersing agents, wetting agents and/or suspending agents. A sterile injectable preparation can also be a sterile injectable solution or suspension in a non-toxic parenterally acceptable diluent or solvent. Among the acceptable vehicles and solvents that can be used are water and isotonic sodium chloride solution. Sterile oils are also conventionally used as solvent or suspending medium.

Suppositories for rectal administration of a pharmaceutical composition of the present invention can be prepared by e.g. mixing the compound with a suitable non-irritating excipient such as cocoa butter, synthetic triglycerides and polyethylene glycols which are solid at room temperature but liquid at rectal temperature such that they will melt in the rectum and release the active agent from said suppositories.

For administration by inhalation, the pharmaceutical composition according to the present invention may be conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Oral dosage forms may be liquid or solid and include e.g. tablets, troches, pills, capsules, powders, effervescent formulations, dragees and granules. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. The oral dosage forms may be formulated to ensure an immediate release of the active agent or a sustained release of the active agent.

### 3. Examples

The following Examples are merely illustrative and shall describe the present invention in a further way. These Examples shall not be construed to limit the present invention thereto.

### Example 1: In Vitro Studies in Cell Lines

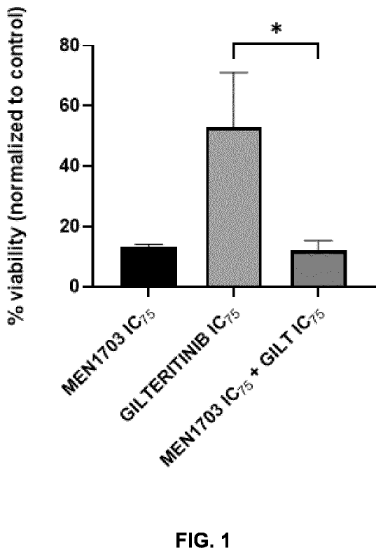

In an in vitro standard cytotoxicity experiment, two AML cell lines (MV4-11 [FLT3 ITD] and MOLM-13 [FLT3 ITD]) were treated for 72 hours with MEN and Gilt (a Type I inhibitor) either as a single agent or in combination. The synergistic effect of the combination was measured according to the Combination Index calculation (Chou T C, 2006). The combination of MEN and Gilt was slightly synergistic in MV4-11 cells at all concentration levels (see Table 1). The combination of MEN and Gilt at the IC75 concentration induced a cytotoxicity significantly different from Gilteritinib-induced cytotoxicity in MV4-11 cells (Student's t test, p=0.0331; see FIG. 1). The combination of MEN and Gilt in MOLM-13 cells did appear to be synergistic at IC25 and IC10 concentration levels (see Table 2).

TABLE 1
in vitro Combination Index in MV4-11 cells

| | MEN | Gilt | |
|---|---|---|---|
| MEN-Gilt | dose level | dose level | Combination Index (CI) |
| dose level | (μM) | (μM) | Mean value (n = 2) |
| IC10 | 0.151 | 0.0022 | 0.94 ± 0.14 |
| IC25 | 0.293 | 0.0042 | 0.76 ± 0.11 |
| IC50 | 0.569 | 0.0079 | 1.00 ± 0.04 |
| IC75 | 1.103 | 0.0140 | 0.87 ± 0.10 |

The combination of MEN+Gilt was moderately synergistic at different concentrations levels (CI≤1). IC75 and IC50 are clinically relevant concentrations (grey rows).

TABLE 2
in vitro Combination Index in MOLM-13 cells

| | MEN | | |
|---|---|---|---|
| | dose | Gilt | Combination Index |
| MEN-Gilt | level | dose level | (CI) |
| dose level | (μM) | (μM) | Mean value (n = 2) |
| IC10 | 0.087 | 0.0014 | 0.77 ± 0.38 |
| IC25 | 0.21 | 0.0040 | 0.8 |
| IC50 | 0.51 | 0.0110 | 1.13 ± 0.04 |
| IC75 | 1.23 | 0.0320 | 1.6 ± 0.30 |

The combination of MEN+Gilt was synergistic at the concentration levels IC10 and IC25 (CI≤1).

### Materials and Methods

### Human Cell Lines

Human acute myeloid leukemia cell lines MV4-11 and MOLM-13 were obtained from DSMZ, Braunschweig, Germany (ACC102 and ACC554). MV4-11 cells were cultured in RPMI 1640 medium (Gibco, Life Technologies, Carlsbad, CA, USA) supplemented with 10% fetal bovine serum (FBS) (Sigma, Saint Louis, CA, USA). MOLM-13 cells were grown in RPMI with 20% FBS. All cells were incubated at 37° C., 5% CO₂, 80% relative humidity.

### Drugs

MEN1703 (alternatively referred to as “Men”, “MEN”, or “SEL24/MEN1703”), in the HCl-salt form (CAS number 2769008-22-) with the IUPAC name 5,6-dibromo-4-nitro-2-(piperidin-4-yl)-1-(propan-2-yl)-1H-1,3-benzodiazole-4-amine hydrochloride (batch no. A/2201/24/1)), synthesized at Aptuit (VR) and Gilteritinib (alternatively referred to as “Gilt”, MedChem Express, Monmouth Junction, NJ, USA) were dissolved in DMSO, divided into aliquots and stored at −80° C. until use. Stock solutions were added at appropriate concentration in culture medium before the addition to cells.

### Cytotoxicity

For cytotoxicity studies cells were seeded at appropriate density (50.000 cells/well) prior to addition of MEN and Gilt at day 0. The concentration range was previously identified in single agent cytotoxicity assays for the same time points in order to include in the combination experiment the IC10, IC25, IC50 and IC75 values for each drug and for each AML cell lines (see Tables 1 to 2). After 72 hours, CellTiter 96 Aqueous One Solution Reagent (MTS) (Promega, Madison. WI, USA) was added to assess the cell viability. Fluorescence was measured 4 h later using Tecan Infinite M200 (Tecan Trading AG, Switzerland), recording absorbance at 490 nm. Quantitative measurement of synergism/antagonism was evaluated with Combination Index (CI) on the Fraction affected (Fa) using CompuSyn (ComboSyn, Inc. Paramus, NJ, USA) (Chou T C, 2006).

### Example 2: In Vivo Xenograft Studies

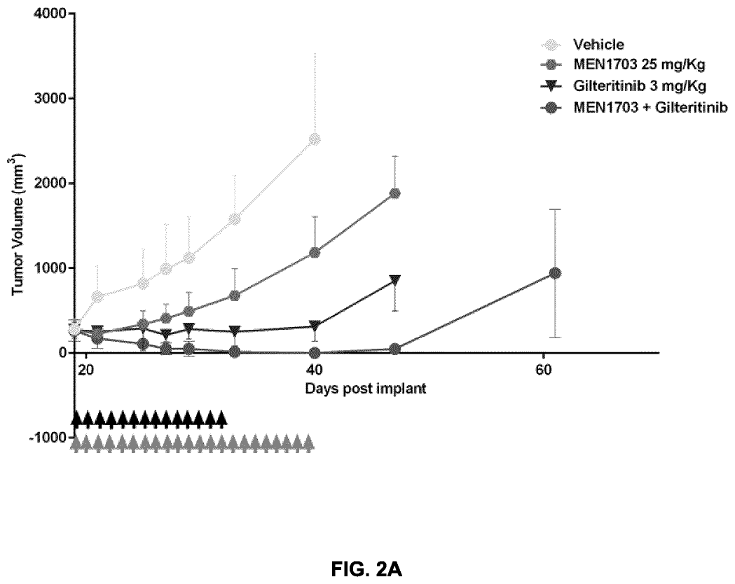

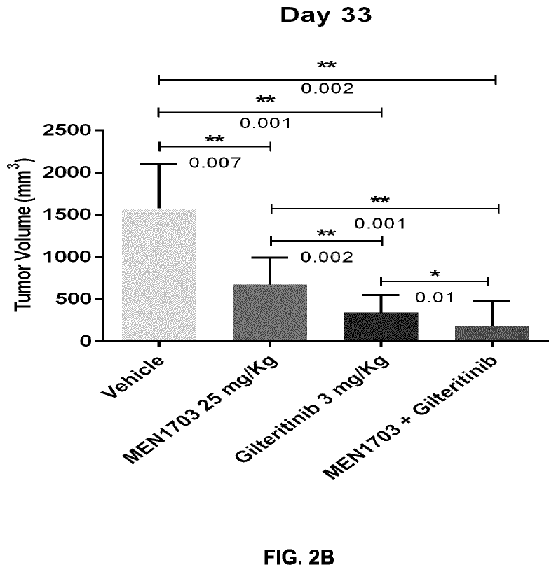

In an in vivo xenograft study with MV4-11 (FLT3 ITD) cells, MEN and Gilteritinib as single agent treatments induced a significant antitumor activity at day 33 showing 57.2% and 84.1% tumor volume inhibition (TVI), respectively, whereas the combination treatment showed 99.2% TVI at day 33 (see FIG. 2A and Table 3). The combination treatment ended at day 33 and the prolonged treatment with Gilteritinib alone thereafter for 8 additional days induced a long-lasting antitumor activity until day 47 in this combination treatment-group. After day 47, regrowth of the tumor masses was observed in this combination treatment group. At day 40, when the Gilteritinib-only treatment was concluded, the single agent treatments with Men and Gilteritinib showed 53.1% and 87.6% TVI, respectively, whereas the combination treatment showed 100% TVI (see FIG. 2A and Table 3).

TABLE 3
in vivo tumor volume inhibition (TVI) at day 33 and day 40, respectively.

| | Dose | Schedule | Route of | TVI % | TVI % |
|---|---|---|---|---|---|
| Compound | (mg/kg) | Q1 = daily | administration | Day 33 | Day 40 |
| MEN | 25 | Q1 × 14 d | per os | 57.2 | 53.1 |
| Gilteritinib | 3 | Q1 × 22 d | per os | 84.1 | 87.6 |
| MEN + Gilt | For each | For each as | For each per | 99.2 | 100 |
| | as above | above | os | | |

Tumor growth inhibition with the combination of MEN+Gilteritinib is improved over the single agent treatments at the end of the respective treatments.

According to the Mann Whitney test, evaluated at day 33, TV reduction was statistically significant when the single agent treatment groups and the combination group was compared to the vehicle group (p-value=0.007; p-value=0.001 and p-value=0.002, respectively), and the combination treatment group was statistically significant when compared to both single agent treatment groups (p-value=0.001, p-value=0.01, see FIG. 2B).

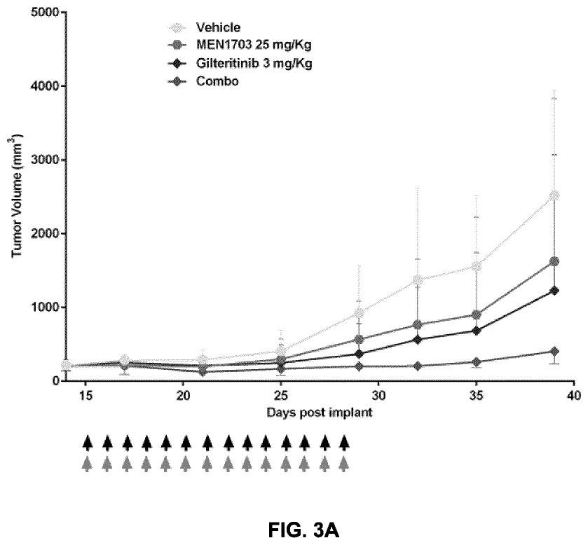

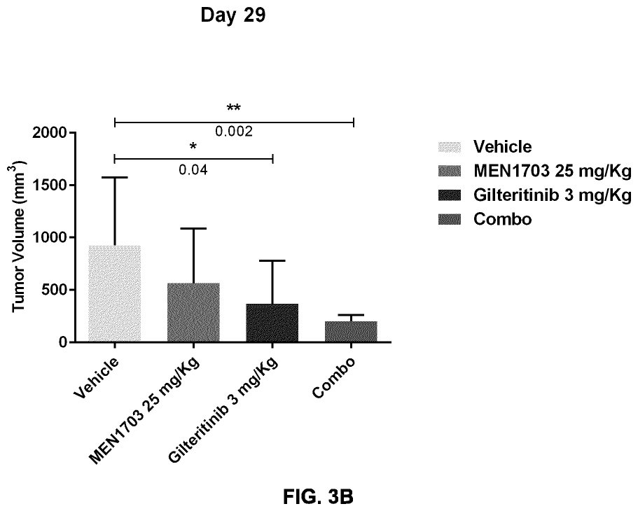

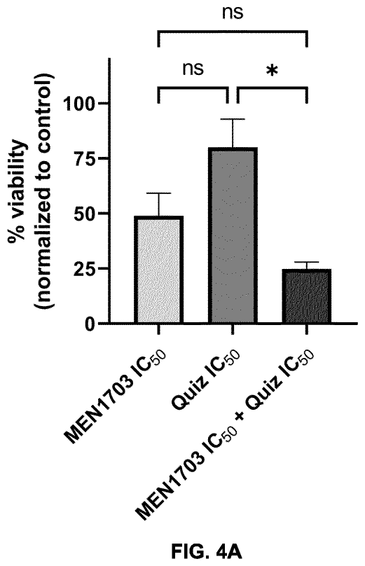

In an in vivo xenograft study with MOLM-13 (FLT3 ITD) cells, MEN and Gilteritinib as single agent treatments induced a significant antitumor activity at day 29 showing 38.8% and 60.1% tumor volume inhibition (TVI), respectively, whereas the combination treatment showed 78.3% TVI at day 29 (see FIG. 3A and Table 4).

TABLE 4
in vivo tumor volume inhibition (TVI) at day 29.

| | Dose | Schedule | Route of | TVI % |
|---|---|---|---|---|
| Compound | (mg/kg) | Q1 = daily | administration | Day 29 |
| MEN | 25 | Q1 × 14 d | per os | 38.8 |
| Gilteritinib | 3 | Q1 × 14 d | per os | 60.1 |
| MEN + Gilt | For each | For each as | For each per | 78.3 |
| | as above | above | os | |

Tumor growth inhibition with the combination of MEN+Gilt is improved over the single agent treatments at the end of the respective treatments.

According to the Mann Whitney test, evaluated at day 29, TV reduction was statistically significant when the gilteritinib-only treatment and the combination treatment were compared to the vehicle group (p-value=0.04 and p-value=0.002, see FIG. 3B).

### Materials and Methods

### Human Cell Line

MV4-11 and MOLM-13 cells were obtained and cultured as described in example 1.

### Drugs

MEN1703 (alternatively referred to as “Men”, “MEN”, or “SEL24/MEN1703”), in the HCl-salt form (CAS number 2769008-22-) with the IUPAC name 5,6-dibromo-4-nitro-2-(piperidin-4-yl)-1-(propan-2-yl)-1H-1,3-benzodiazole-4-amine hydrochloride (batch 76608X, synthesized at MENARINI RICERCHE SpA Pisa) was dissolved in sterile water. Gilteritinib (MedChem Express, Monmouth Junction, NJ, USA) was dissolved in a solution of carboxymethylcellulose (0.5%).

### MV4-11 Model

For the AML xenograft model, 10×10⁶ MV4-11 cells were re-suspended in 0.2 ml of BME type III (Trevigen) at 5.6 mg/ml+DPBS (1:1) and then injected subcutaneously into the right flank of 6-8 weeks old female SCID mice (Charles River, Calco, Italy).

After injection, mice were maintained in micro isolator cages under continuously monitored environmental conditions. Drinking water and specific sterilized diet (VRF1, Charles River) were supplied ad libitum. Environmental conditions, as well as the procedures for housing and handling the animals, complied with the UKCCCR guideline (Workman P et al., 2010) and the European Convention for the protection of vertebrate animals used for experimental and other scientific purposes (Directive 2010/63/EU; 2010). Twice a week, tumor growth and body weight were evaluated and recorded.

The outcome evaluation was carried out as follows: tumor volumes were measured by caliper, and tumor masses were calculated using the following formula: [length (mm)×width² (mm)×d]/2, assuming density, d=1 mg/mm³ for tumor tissue (Teicher B. Totowa, 1997). When the average tumor volume reached 200-300 mm³ (corresponding to day 19 in FIG. 2A) animals were randomly assigned into four groups (6-7 mice/group) and the following treatments were administered starting from day 19: Group I received vehicle carboxymethyl cellulose 0.5% (Gilteritinib diluent solution) perosonce daily for 22 days, i.e. until the end of the administration period (i.e. until day 40, see FIG. 2A), group II received MEN at 25 mg/kg perosonce daily for 14 days (i.e. until day 33, see FIG. 2A), group III received Gilteritinib at 3 mg/kg per os once daily for 22 days, i.e. until day 40, see FIG. 2A), and group IV received the combination of MEN and Gilteritinib, wherein the afore-mentioned dosages and regimen of the active agents were used in the combinations as well.

The treatment efficacy was assessed as TVI % in treated versus control mice, using the following formula: TVI %=(1−Volume Average of treated tumor mass/Volume Average of control tumor)*100.

Mice were sacrificed when tumors reached a volume of around 10% of total body weight or when mice's body weight decreased by more than 20% compared to control animals for 7 days or more. Animals were euthanized with carbon dioxide exposure according to the standard procedures (Annex IV of Directive 2010/63/EU; 2010).

### MOLM-13 Model

For the AML xenograft model, 10×10⁶ MOLM-13 cells were re-suspended in 0.2 ml of BME type III (Trevigen) at 5.6 mg/ml+DPBS (1:1) and then injected subcutaneously into the right flank of 6-8 weeks old female CD-1 mice (Charles River, Calco, Italy).

After injection, mice were maintained in micro isolator cages under continuously monitored environmental conditions. Drinking water and specific sterilized diet (VRF1, Charles River) were supplied ad libitum. Environmental conditions, as well as the procedures for housing and handling the animals, complied with the UKCCCR guideline (Workman P et al., 2010) and the European Convention for the protection of vertebrate animals used for experimental and other scientific purposes (Directive 2010/63/EU; 2010). Twice a week, tumor growth and body weight were evaluated and recorded.

The outcome evaluation was carried out as follows: tumor volumes were measured by caliper, and tumor masses were calculated using the following formula: [length (mm)×width² (mm)×d]/2, assuming density, d=1 mg/mm³ for tumor tissue (Teicher B. Totowa, 1997). When the average tumor volume reached 200-300 mm³ (corresponding to day 15 in FIG. 3A) animals were randomly assigned into four groups (6-9 mice/group) and the following treatments were administered starting from day 15: Group I received vehicle carboxymethyl cellulose 0.5% (Gilteritinib diluent solution) perosonce daily for 14 days (i.e. until and including day 28, see FIG. 3A), group II received MEN at 25 mg/kg per os once daily for 14 days (i.e. until and including day 28, see FIG. 3A), group III received Gilteritinib at 3 mg/kg per os once daily for 14 days (i.e. until and including day 28, see FIG. 3A), and group IV received the combination of MEN and Gilteritinib, wherein the afore-mentioned dosages and regimen of the active agents were used in the combinations as well.

The treatment efficacy was assessed as TVI % in treated versus control mice, using the following formula: TVI %=(1−Volume Average of treated tumor mass/Volume Average of control tumor)*100.

Mice were sacrificed when tumors reached a volume of around 10% of total body weight or when mice's body weight decreased by more than 20% compared to control animals for 7 days or more. Animals were euthanized with carbon dioxide exposure according to the standard procedures (Annex IV of Directive 2010/63/EU; 2010).

### Statistical Analyses

GraphPad Prism software (GraphPAD Software Inc., California) was used for statistical analysis. Statistical differences were considered to be significant at p-value<0.05 using the two-tailed Mann-Whitney rank test. In vivo data (FIGS. 2 to 3) are presented as mean, with value for each group represented as a symbol of different shapes and lines of different colors.

### Example 3: In Vitro Studies in Cell Line MOLM-13

In an in vitro standard cytotoxicity experiment, the AML cell line MOLM-13 was treated for 72 hours with MEN and Quizartinib (a Type II inhibitor) either as a single agent or in combination. The synergistic effect of the combination was measured according to the Combination Index calculation (Chou T C, 2006).

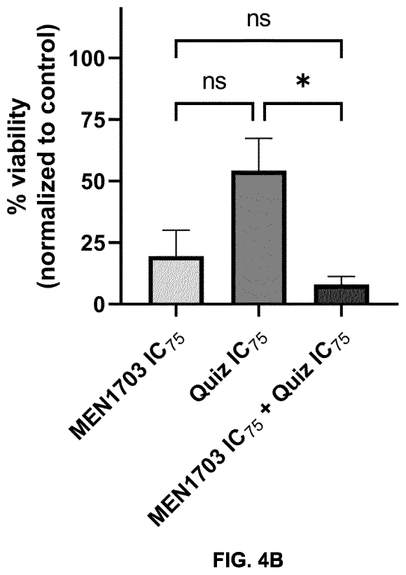

The synergistic effect of the combination of MEN and Quizartinib in MOLM.13 cells is shown in Table 5 (see Table 5, CI<1.0). The combination of MEN and Quizartinib at the IC50 concentrations and the IC75 concentrations induced a cytotoxicity significantly different from Quizartinib-induced cytotoxicity in MOLM-13 cells (Tukey's multiple comparison one-way ANOVA test; p<0.05*, see FIGS. 4A and B).

TABLE 5
in vitro Combination Index in MOLM-13 cells

| | | Combination Index | | | |
|---|---|---|---|---|---|
| | | MEN IC10 | MEN IC25 | MEN IC50 | MEN IC75 |
| | | 0.08729 μM | 0.3222 μM | 0.5104 μM | 1.234 μM |
| QUIZ IC10 | 0.000374 μM | 1.31 | 1.36 | 0.84 | 0.58 |
| QUIZ IC25 | 0.000595 μM | 1.26 | 0.95 | 0.84 | 0.58 |
| QUIZ IC50 | 0.000946 μM | 0.73 | 0.75 | 0.69 | 0.48 |
| QUIZ IC75 | 0.001504 μM | 0.68 | 0.67 | 0.61 | 0.47 |

### Materials and Methods

### Human Cell Line

Human acute myeloid leukemia cell line MOLM-13 was obtained from DSMZ, Braunschweig, Germany (ACC554). MOLM-13 cells were grown in RPMI with 20% FBS and incubated at 37° C., 5% CO₂, 80% relative humidity.

### Drugs

MEN1703 (alternatively referred to as “Men”, “MEN”, “MEN1703” or “SEL24/MEN1703”), in the HCl-salt form (CAS number 2769008-22-) with the IUPAC name 5,6-dibromo-4-nitro-2-(piperidin-4-yl)-1-(propan-2-yl)-1H-1,3-benzodiazole-4-amine hydrochloride (batch no. A/2201/24/1)), synthesized at Aptuit (VR) and Quizartinib (alternatively referred to as “Quiz”, MedChem Express, Monmouth Junction, NJ, USA, batch no. 20907) were dissolved in DMSO, divided into aliquots and stored at −80° C. until use. Stock solutions were added at appropriate concentration in culture medium before the addition to cells.

### Cytotoxicity

For cytotoxicity studies cells were seeded at appropriate density (50.000 cells/well) prior to addition of MEN and Quiz at day 0. The concentration range was previously identified in single agent cytotoxicity assays for the same time points in order to include in the combination experiment the IC10, IC25, IC50 and IC75 values for each drug (see Table 5). After 72 hours, CellTiter 96 Aqueous One Solution Reagent (MTS) (Promega, Madison. WI, USA) was added to assess the cell viability. Fluorescence was measured 4 h later using Tecan Infinite M200 (Tecan Trading AG, Switzerland), recording absorbance at 490 nm. Quantitative measurement of synergism/antagonism was evaluated with Combination Index (CI) on the Fraction affected (Fa) using CompuSyn (ComboSyn, Inc. Paramus, NJ, USA) (Chou T C, 2006).

### Example 4: In Vitro Studies in Cell Line KG-1

In an in vitro standard cytotoxicity experiment, the AML cell line KG-1 (FLT wildtype) was treated for 72 hours with MEN and Gilt (a Type I inhibitor) either as a single agent or in combination. The synergistic effect of the combination was measured according to the Combination Index calculation (Chou T C, 2006).

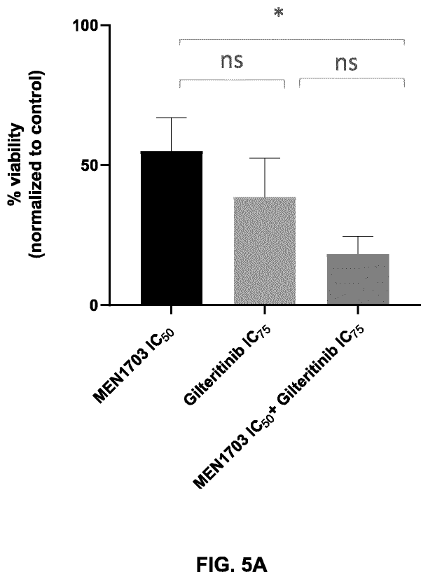

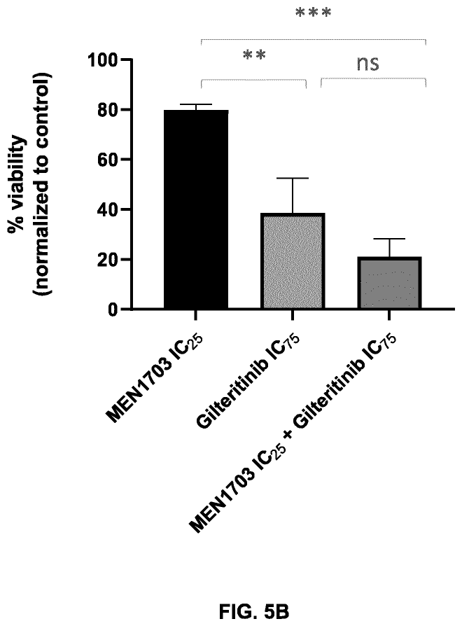

The synergistic effect of the combination of MEN and Gilt in KG-1 cells is shown in Table 6 (see Table 6, CI<1.0). The combination of MEN at the IC50 concentration and Gilt at the IC75 concentration induced a cytotoxicity significantly different from MEN1703-induced cytotoxicity in KG-1 cells (Tukey's multiple comparison one-way ANOVA test; p<0.0158*, see FIG. 5A). The combination of MEN at the IC25 concentration and Gilt at the IC75 concentration induced a cytotoxicity significantly different from MEN1703-induced cytotoxicity in KG-1 cells (Tukey's multiple comparison one-way ANOVA test; p<0.0005***, see FIG. 5B).

TABLE 6
in vitro Combination Index in KG-1 cells

| | | Combination Index | | | |
|---|---|---|---|---|---|
| | | MEN IC10 | MEN IC25 | MEN IC50 | MEN IC75 |
| | | 0.05748 μM | 0.203 μM | 0.7166 μM | 2.530 μM |
| Gilt IC10 | 0.03534 μM | 1.98 | 1.33 | 0.53 | 0.64 |
| Gilt IC25 | 0.15020 μM | 3.38 | 0.97 | 0.38 | 0.69 |
| Gilt IC50 | 0.63820 μM | 1.00 | 0.44 | 0.25 | 0.57 |
| Gilt IC75 | 2.7120 μM | 0.39 | 0.21 | 0.19 | 0.31 |

### Materials and Methods

### Human Cell Lines

Human acute myeloid leukemia cell line KG-1 was obtained from DSMZ, Braunschweig, Germany (ACC14). KG-1 cells were grown in RPMI+10% FBS and incubated at 37° C., 5% CO₂, 80% relative humidity.

### Drugs

MEN1703 (alternatively referred to as “Men”, “MEN”, or “SEL24/MEN1703”), in the HCl-salt form (GAS number 2769008-22-) with the IUPAC name 5,6-dibromo-4-nitro-2-(piperidin-4-yl)-1-(propan-2-yl)-1H-1,3-benzodiazole-4-amine hydrochloride (batch no. A/2201/24/1)), synthesized at Aptuit (VR) and Gilteritinib (alternatively referred to as “Gilt”, MedChem Express, Monmouth Junction, NJ, USA) were dissolved in DMSO, divided into aliquots and stored at −80° C. until use. Stock solutions were added at appropriate concentration in culture medium before the addition to cells.

### Cytotoxicity

For cytotoxicity studies cells were seeded at appropriate density (50.000 cells/well) prior to addition of MEN and Gilt at day 0. The concentration range was previously identified in single agent cytotoxicity assays for the same time points in order to include in the combination experiment the IC10, IC25, IC50 and IC75 values for each drug (see Table 6). After 72 hours, CellTiter 96 Aqueous One Solution Reagent (MTS) (Promega, Madison. WI, USA) was added to assess the cell viability. Fluorescence was measured 4 h later using Tecan Infinite M200 (Tecan Trading AG, Switzerland), recording absorbance at 490 nm. Quantitative measurement of synergism/antagonism was evaluated with Combination Index (CI) on the Fraction affected (Fa) using CompuSyn (ComboSyn, Inc. Paramus, NJ, USA) (Chou T C, 2006).

### 4. References

Chou T C, Theoretical basis, experimental design, and computerized simulation of synergism and antagonism in drug combination studies, Pharmacol Rev, 2006
Czardybon W. et al., A novel, dual pan-PIM/FLT3 inhibitor SEL24 exhibits broad therapeutic potential in acute myeloid leukemia. Oncotarget, 2018, 9(24), 16917-16931
Directive 2010/63/Eu of The European Parliament And Of The Council On The Protection Of Animals Used For Scientific Purposes, 22 Sep. 2010
Kennedy V E and Smith C C, FLT3 mutations in acute myeloid leukemia: key concepts and emerging controversies, Frontiers in Oncology, 2020, 10:612880
Levis and Perl, Gilteritinib: potent targeting of FLT3 mutations in AML, Blood advances, 2020, 4(6), 1178-1191
Nakao M, et al., Internal tandem duplication of the flt3 gene found in acute myeloid leukemia. Leukemia, 1996, 10(12), 1911-1918
Qiao et al. The combination of CUDC-907 and gilteritinib shows promising in vitro and in vivo antileukemic activity against FLT3-ITD AML, 2021, Blood Cancer Journal, 11:111
Teicher B. Totowa. Anticancer Drug Development Guide. New Jersey: Humana Press, 1997
Workman P., et al., Guidelines for The Welfare And Use Of Animals In Cancer Research. British Journal Of Cancer; 2010, 102, 1555-1577
Yuan et al., Dual FLT3 inhibitors: Against the drug resistance of acute myeloid leukemia in recent decade. European Journal of Medicinal Chemistry, 2019, 178, 468-483
Zhang et al., Rapid and efficient response to gilteritinib and venetoclax-based therapy in two AML patients with FLT3-ITD mutation unresponsive to venetoclax plus azacitidine, Onco Targets and Therapy, 2022: 15, 159-164

1. A combination of (i) a FLT3-inhibitor and (ii) SEL24/MEN1703 for use as medicament.

2. The combination for use according to claim 1, wherein the FLT3-inhibitor is selected from the group consisting of Gilteritinib, Quizartinib, Midostaurin, and combinations thereof.

3. A combination of (i) a FLT3-inhibitor and (ii) SEL24/MEN1703 for use in the treatment of a patient suffering from cancer.

4. The combination for use according to claim 3, wherein the cancer is a hematological cancer.

5. The combination for use according to claim 3 or 4, wherein the cancer is acute myeloid leukemia (AML).

6. The combination for use according to any one of the preceding claims, wherein SEL24/MEN1703 is administered at a daily dose of about 50 mg to about 150 mg.

7. The combination for use according to any one of claims 2 to 6, wherein Gilteritinib is administered at a daily dose of about 40 mg to about 400 mg.

8. The combination for use according to any one of claims 2 to 7, wherein Quizartinib is administered at a daily dose of about 10 mg to about 50 mg.

9. The combination for use according to any one of claims 2 to 8, wherein Midostaurin is administered at a daily dose of about 20 mg to about 80 mg.

10. The combination for use according to any one of the preceding claims, wherein (i) and (ii) are administered as separate dosage forms.

11. The combination for use according to any one of the preceding claims, wherein (i) and (ii) are administered orally.

12. A kit of dosage forms comprising (i) a dosage form comprising a FLT3-inhibitor and (ii) a dosage form comprising SEL24/MEN1703.

13. The kit according to claim 12, wherein the FLT3-inhibitor is selected from the group consisting of Gilteritinib, Quizartinib, Midostaurin, and combinations thereof.

14. A dosage form comprising (i) a FLT3-inhibitor and (ii) SEL24/MEN1703.

15. The dosage form according to claim 14, wherein the FLT3-inhibitor is selected from the group consisting of Gilteritinib, Quizartinib, Midostaurin, and combinations thereof.
